(19) United States
(12) Patent Application Publication
(10) Pub. No.: US 2012/0207722 A1
(43) Pub. Date: Aug. 16, 2012

(54) Use Of Adipose Tissue Cells For Initiating The Formation Of A Functional Vascular Network

(75) Inventors: Louis Casteilla, Escalquens (FR); Jean-Sébastien Silvestre, Paris (FR); Valérie Planat-Benard, Montrabe (FR); Bernard Levy, Paris (FR); Luc Penicaud, Toulouse (FR); Alain Tedgui, Paris (FR)

(73) Assignees: CENTRE NATIONAL DE LA RECHERCHE SCIENTIFIQUE, PARIS (FR); INSTITUT NATIONAL DE LA SANTE ET DE LA RECHERCHE MEDICALE, PARIS (FR); UNIVERSITE PARIS VII (DENIS DIDEROT), PARIS (FR); UNIVERSITE PAUL SABATIER, TOULOUSE (FR)

(21) Appl. No.: 13/369,245

(22) Filed: Feb. 8, 2012

Related U.S. Application Data
(62) Division of application No. 10/570,458, filed on Nov. 7, 2006, now abandoned.

(30) Foreign Application Priority Data
Sep. 5, 2003 (FR) 0310504

(51) Int. Cl. A61K 35/12; A61P 9/10

(52) U.S. Cl. 424/93.21; 424/93.7

(57) ABSTRACT

This invention relates to the use of cells of a medullary or extra-medullary white adipose tissue, in particular of an extra-medullary stromal vascular fraction (SVF) and/or mature dedifferentiated adipocytes of any origin for initiating the formation of a functional vascularisation.

This application is a divisional of U.S. application Ser. No. 10/570,458, filed Nov. 7, 2006, now abandoned, which is a U.S. National Phase Application Under 35 U.S.C. §371 of PCT Application No. PCT/FR04/02258 filed Sep. 6, 2004, which claims priority to French Application No. 03 10504, filed Sep. 5, 2003, each of which is incorporated by reference in its entirety for all purposes.

### BACKGROUND OF THE INVENTION

The present invention relates to the use of cells from medullary or extramedullary white adipose tissue, and in particular from the extramedullary stromal-vascular fraction (SVF) and/or of mature dedifferentiated adipocytes of any origin, for inducing the formation of a functional vascularization.

There exists a need, in particular in western societies, for effective therapeutic means which stimulate neovascularization, for limiting the complications associated with ischemic pathologies and/or promoting tissue regeneration.

Up until now, the therapeutic strategies proposed for limiting the harmful effects of ischemia have called upon the stimulation of the growth and of the remodeling of the vessels at the very site of the ischemia and/or upon the transplantation of endothelial progenitor cells. For obtaining endothelial cells capable of effectively allowing revascularization of an area that has been rendered ischemic, the methods proposed up until now have been essentially based on obtaining mature endothelial cells from circulating adult endothelial progenitor cells. These human mononuclear cells of hematopoietic origin, derived from the monocyte/macrophage line (CD45+, CD14+), are isolated from the bone marrow (BM-MNCs for bone marrow-mononuclear cells) or from peripheral blood, in the presence of angiogenic growth factors (20; 21; 27; 38; 39).

More particularly, it has been shown that the transplantation of BM-MNCs (bone marrow-mononuclear cells) effectively stimulates neovascularization in experimental ischemias and leads to a significant improvement and long-term survival of the lesioned tissues (26; 27). The trials carried out in humans show the potential of such a therapy for limiting the progression of the disease (28-32).

However, the low percentage and the difficulty in ex vivo expansion of these endothelial progenitor cells and the functional deterioration of these cells, observed under pathological conditions, constitute a major limitation to their use in the treatment of ischemia.

Consequently, there exists therefore a real need to provide a source of cells that form a homogeneous cell population capable of differentiating into mature endothelial cells that can be used in particular in the context of the repair of tissues damaged by ischemia, which is simple to obtain and effective.

Adipose tissue exists in various forms in mammals: extramedullary white adipose tissue, which represents the main storage organ of the organism, medullary white adipose tissue, the exact role of which is not known, and thermogenic brown adipose tissue.

Because of its considerable potential for expansion which persists throughout the individual's life, white adipose tissue in adults constitutes a source of abundant cells that are easy to obtain.

This white adipose tissue consists of two cell fractions:
an adipocyte fraction which represents 30% to 60% of the cells of the adipose tissue and is characterized by the accumulation of triglycerides (floating cell fraction). This fraction is very predominantly (99%) composed of differentiated adipocytes and a few contaminating macrophages, rich in lipid droplets, and
a non-adipocyte fraction, called stromal-vascular fraction (SVF) comprising some blood cells, some mature endothelial cells (cells of the micro-vascular endothelium: CD31⁺, CD144⁺), pericytes, fibroblasts and pluripotent stem cells.

It has been shown that the stromal-vascular fraction, conventionally used to study the differentiation of preadipocytes into mature adipocytes, is a source of pluripotent stem cells comprising, in addition to adipocyte progenitors (preadipocytes), only hematopoietic and neurogenic pregenitors, and also mesenchymal stem cells capable of differentiating into osteogenic, chondrogenic and myogenic lines (10; 11; 12; 13; PCT international application WO 02/055678 and American application US 2003/0082152).

These two cell fractions can be separated by virtue of their difference in density, according to methods such as those described by Björntorp et al. (14).

White adipose tissue possesses unique angiogenic properties resulting from the effect, on differentiated vascular endothelial cells, of pro-angiogenic factors produced by the adipocytes (Bouloumié et al., Ann. Endocrin., 2002, 63, 91-95; Wang et al., Horm. Metab. Res., 2003, 35, 211-216) and the cells of the stromal-vascular fraction (Rehman et al., Journal of the American College of Cardiology, 2003, 41, 6 supplement A, 3008A). These angiogenic properties, which probably play a role in the metabolic activity in the expansion of adipose tissue, have applications in autologous cell therapy, for promoting angiogenesis in a post-traumatic or pathological (post-ischemic) context. Thus, the injection of autologous adipose tissue is commonly practiced in surgery, to promote the revascularization of transplants and the reconstruction of soft tissues (Bouloumié et al., Wang et al., mentioned above). Furthermore, it has also been recommended to administer the autologous stromal-vascular fraction, in order to promote angiogenesis, in the treatment of coronary disease (Rehman et al., mentioned above).

However, neovascularization results not only from the effect of pro-angiogenic factors on the endothelial cells of the preexisting vessels (angiogenesis), but also from the production and the incorporation, into the forming vessels, of differentiated endothelial cells produced from endothelial progenitor cells (vasculogenesis).

Such endothelial progenitor cells have not been isolated from adipose tissue, and in particular from the stromal-vascular fraction containing pluripotent cells.

### SUMMARY OF THE INVENTION

In this context, the inventors have isolated a homogeneous subpopulation of cells of medullary or extramedullary adipose tissue (easy to obtain (liposuction, for example)), capable of differentiating into mature endothelial cells which make it possible to obtain total or partial reconstruction of a functional vascular network.

Consequently, a subject of the present invention is the use of cells of medullary or extramedullary white adipose tissue forming homogeneous subpopulations, which express at least the surface antigens CD13 and HLA ABC(CD13⁺, HLA ABC⁺), for preparing a medicinal product intended for the total or partial reconstruction of a functional vascular network, in particular in the context of an ischemia.

According to a first advantageous embodiment of said use, said cells forming homogeneous subpopulations also express the surface antigen CD34.

According to a second advantageous embodiment of said use, said adipose tissue cells are represented by a homogeneous subpopulation of cells of the extramedullary stromal-vascular fraction (hereinafter referred to as SVF-CULT), obtainable by limited cellular expansion in culture.

According to an advantageous arrangement of this embodiment, said homogeneous subpopulation of cells of the extramedullary stromal-vascular fraction is obtainable by a limited cellular expansion with less than 10 successive passages of said cells.

Thus, surprisingly, a limited cellular expansion, because of the number of successive passages limited to 10 at most, promotes the proliferation of a homogeneous population of cells which have surface antigens that are characteristic of cells with pro-angiogenic potential, but which do not have any surface marker characteristic of hematopoietic cells including those of the monocyte/macrophage line or differentiated endothelial cells.

Also surprisingly, such cells are obtained by culturing in a minimum medium, such as a DMEM medium comprising 10% of fetal or newborn calf serum, for example.

Specific conditions, that initiate more rapidly pro-angiogenic characteristics, can also be used. They are specified hereinafter (see method for selecting adipose tissue cells).

According to a third advantageous embodiment of said use, said adipose tissue cells are represented by a homogeneous subpopulation of mature dedifferentiated adipocytes (hereinafter referred to as DDACs).

The dedifferentiated adipocytes are in particular obtained under the conditions described in R. Negrel et al. (17) or in M. Shigematsu et al. (19).

Thus, by limited expansion of the extramedullary stromal-vascular fraction or by dedifferentiation of mature adipocytes, subpopulations of cells expressing at least the abovementioned surface antigens, i.e.: CD13, HLA ABC(CD13⁺, HLA ABC⁺), are obtained. The CD34 surface antigen, which is present in freshly isolated cells, can gradually disappear in the course of the successive passages in culture. On the other hand, these cells do not express in particular the following surface antigens: CD45, CD14, CD31 and CD144 (CD45⁻, CD14⁻, CD31⁻ and CD144⁻). The subpopulations of cells expressed in at least the above-mentioned surface antigens are capable of differentiating into functional endothelial cells expressing the CD31 and CD144 surface antigens.

According to a fourth advantageous embodiment of said use, said adipose tissue cells forming homogeneous subpopulations, which express at least the following surface antigens: CD13⁺, HLA ABC⁺, are associated with a solid or semi-solid polymeric support.

According to one advantageous arrangement of this embodiment, said solid polymeric support is preferably a reconstituted basal membrane matrix comprising at least one of the following elements: collagen, laminin and proteoglycans, or a reconstituted extracellular matrix comprising one of the following elements: fibronectin, collagen, laminin and thrombospondin. Said support can also comprise enzymes that degrade said matrices, and also enzymatic inhibitors and growth factors. By way of example of matrices that are particularly suitable, mention may be made of the Matrigel® matrices (Becton Dickinson; 40).

According to another advantageous arrangement of this embodiment, said semi-solid polymeric support is preferably a cellulose derivative, and in particular methylcellulose.

In accordance with the invention, said cells can also be genetically modified. Thus:
they can comprise at least one mutation of an autologous gene, or
they can contain at least one copy of a heterologous gene.

Said genetically modified cells are preferably of human origin.

A subject of the present invention is also the use of a composition containing cells of medullary or extramedullary white adipose tissue forming homogeneous subpopulations, which express at least the following surface antigens: CD13⁺, HLA ABC⁺ as defined above, and at least one vehicle and/or one support that is suitable for parenteral or intra-site administration (in situ in the damaged organ), for preparing a medicinal product intended for the total or partial reconstruction of a functional vascular network.

A subject of the present invention is also a pharmaceutical composition containing cells of medullary or extramedullary white adipose tissue forming homogeneous subpopulations, which express at least the surface antigens CD13 and HLA ABC as defined above, said cells being associated with a solid or semi-solid polymeric support as defined above, and at least one vehicle and/or one support that is suitable for parenteral or intra-site administration.

The cells as defined in the present invention are useful for the treatment of any ischemic pathology, in particular cardiovascular pathologies, such as atherosclerosis. In fact, the factor triggering ischemia in a patient suffering from arteritis is the rupture of an atheroma plaque and the formation of a thrombus.

These cells are active regardless of the nature of the tissue rendered ischemic and can be used for the treatment of an ischemia affecting a tissue such as, in particular, the brain, the pancreas, the liver, the muscle and the heart.

These cells are active regardless of the route of administration; they can be administered in particular generally (intramuscularly, intraperitoneally or intravenously) or directly into the damaged tissue.

A subject of the present invention is also a method for culturing cells of medullary or extramedullary white adipose tissue forming homogeneous subpopulations, which express at least the surface antigens CD13 and HLA ABC, which method is characterized in that it comprises at least the following steps:
limited cellular expansion of cells of said adipose tissue (cells of the extramedullary stromal-vascular fraction or mature dedifferentiated adipocytes), with less than 10 successive passages of said cells, on a suitable solid culture support, in a medium comprising at least one growth factor capable of stimulating the formation of endothelial cells and, optionally, at least one suitable cytokine;
continuous or transient modification of the oxygen environment of the culture, and
continuous or transient modification of the redox equilibrium of said cells or of the production of active oxygen species by said cells, by the addition of pro- or antioxidant molecules to the extracellular or intracellular medium.

In accordance with the invention:
said cells of medullary or extramedullary white adipose tissue forming homogeneous subpopulations, which express at least the surface antigens CD13 and HLA ABC, consist of the extramedullary stromal-vascular fraction or of dedifferentiated adipocytes, and
said culture medium is preferably a liquid culture medium.

According to an advantageous embodiment of said method, the growth factor capable of stimulating the formation of endothelial cells is in particular VEGF, preferably at a concentration of approximately 10 ng/ml.

According to another advantageous embodiment of said method, the oxygen environment of the culture is at 1%; from a few hours to a few days.

The pro- or antioxidant molecules are in particular selected from the group consisting of:
inhibitors and/or activators of mitochondrial function, and in particular antimycin, preferably at a concentration of between 1 and 1000 nM, preferably 1 to 100 nM, rotenone at a concentration between 1 and 100 nM, oligomycin at a concentration of between a few ng and a few μg/ml, coenzyme Q, nucleotides or any other equivalent molecule, and carbonyl cyanide m-chlorophenylhydrazone, and
antioxidants selected from the group consisting of trolox, pyrrolidine dithiocarbamate, N-acetylcysteine, manganese (III) tetrakis(4-benzoic acid)porphyrin or any other equivalent molecule.

A subject of the present invention is also a method for screening for molecules that are active on differentiated endothelial cells, which method is characterized in that it comprises at least the following steps:
culturing cells of medullary or extramedullary white adipose tissue forming homogeneous subpopulations, which express at least the surface antigens CD13 and HLA ABC, as defined above, in a semi-solid polymeric culture medium,
bringing the differentiated endothelial cells thus obtained into contact with a library of molecules to be tested,
identifying and selecting the molecules that are active on said cells.

The method according to the invention is useful for screening for both novel chemical molecules and the product of novel genes potentially active on the differentiated endothelial cells.

According to an advantageous embodiment of said screening method, the step consisting in culturing in a solid medium is preceded by preculturing under conditions that make it possible to increase the proangiogenic potential of said cells, as defined above.

According to another advantageous embodiment of said screening method, the step consisting in culturing in a semi-solid medium is carried out under conditions that make it possible to increase the pro-angiogenic potential of said cells, as defined above.

Besides the above arrangements, the invention also comprises other arrangements, which will emerge from the following description, which refers to examples of implementation of the method that is the subject of the present invention and also to the attached drawings.

### BRIEF DESCRIPTION OF THE DRAWINGS

FIG. 1 illustrates the angiogenic properties of the mouse cells of the extramedullary stromal-vascular fraction (SVF) cultured under the conditions of the invention, after injection thereof into the hind limb rendered ischemic, in comparison with bone marrow mononuclear cells (BM-MNCs); in the interest of greater clarity, the cells obtained by culturing this fraction under the conditions of the invention are called SVF-CULT cells in the remainder of the examples.

FIG. 1a) Analysis of the vessel density by microangiography. Left panel: microangiography representative of a right hind limb rendered ischemic (Isch) and of a left hind limb not rendered ischemic (N-Isch), 15 days after femoral occlusion. The arrows indicate the ligatured ends of the femoral artery. Right panel: angiographic score in the limb rendered ischemic and treated, compared with the limb not rendered ischemic.

FIG. 1b) in vivo analysis of blood flow in the hind limb by laser Doppler perfusion imaging, 15 days after occlusion of the femoral artery. Left panel: image of blood flow indicating normal perfusion (represented in black), in the limb not rendered ischemic and the limb rendered ischemic and treated with the SCF-CULT cells, and also a clear reduction in blood flow in the hind limb rendered ischemic and treated with PBS. Right panel: measurement of blood flow in the limb rendered ischemic and treated, compared with the limb not rendered ischemic.

FIG. 1c) analysis of the capillary density by immunolabeling of total fibronectin. Right panel: photomicrographs representative of sections of muscle rendered ischemic, 15 days after femoral occlusion. The capillaries, indicated by arrows, appear in white and the myocytes in black. Right panel: measurement of capillary density in the limb rendered ischemic and treated, compared with the limb not rendered ischemic.

PBS: Mice treated with PBS. SVF: Mice treated with the SVF-CULT cells. BM-MNC: Mice treated with bone marrow cells. The values represent the mean±standard deviation, n=6 per group; **P<0.01;

FIG. 2 shows that the expansion of a heterogeneous population of human cells of the stromal-vascular fraction (extemporaneous preparation obtained before culture, hereinafter referred to as SVF-EXT) under the conditions of the invention effectively promotes the appearance of a homogeneous cell population (SVF-CULT):

FIGS. 2a) and 2b): dispersion diagrams for the SVF-EXT cells and for the SVF-CULT cells; these diagrams comprise, along the x-axis, an estimation of the cell size (FSC height: forward scatter height) and, along the y-axis, the granulocity of the cells (SSC height: side scatter height).

FIGS. 2c) and 2d): identification of the CD45, CD14 and CD144 antigens, characteristic respectively of hematopoietic cells (CD45), of monocytes/macrophages (CD14) and of mature endothelial cells (CD144) in a heterogeneous population of SVF-EXT cells (white columns) in comparison with SVF-CULT cells (black columns);

FIG. 3 shows that the human SVF-CULT cells possess the functional and antigenic properties of endothelial cell precursors, after injection thereof into the hind limb rendered ischemic:

FIGS. 3a) and 3b) the injection of human SVF-CULT cells significantly increases the angiographic score and the blood flow measured in vivo by laser Doppler perfusion imaging, in the right hind limb rendered ischemic and receiving a graft, when compared with the left hind limb not rendered ischemic and not receiving a graft (PBS group) (*P<0.05);

FIG. 3c) 15 days after the injection of human SVF-CULT cells, the antibody directed specifically against an isoform of the human CD31 marker labels numerous CD31-positive cells (indicated with black arrows) which border functional vessels containing erythrocytes (indicated with a gray arrow inside a vessel) (×1000);

FIG. 4 illustrates the differentiation of SVF-CULT cells into endothelial cells under in vitro conditions or in a Matrigel® matrix grafted in vivo:

FIG. 4a) differentiation of SVF-CULT cells into adipocytes in an adipogenic medium (×200);

FIG. 4b) formation of branchy alignments and of tubular-type structures spontaneously when the SVF-CULT cells are seeded in a medium containing methylcellulose (×200);

FIGS. 4c) and 4d) labeling of the SVF-CULT cells seeded in a medium containing methylcelluose, with antibodies directed, respectively, against an isoform of the human CD31 marker and against the vWF (von Willebrand factor) marker; the formation of branchy alignments is noted (×600);

FIGS. 4e) and 4f) formation, in a Matrigel® matrix containing the SVF-CULT cells and grafted in vivo, of tubular-type structures (indicated with black arrows); erythrocytes were also observed in the tubular-type structures (indicated with gray arrows);

FIGS. 4g) and 4h) labeling of the SVF-CULT cells bordering the tubular-type structures of the Matrigel® inclusion, with an antibody against an isoform of the human CD31 marker (×400 and 1000);

FIG. 5 illustrates the dedifferentiation of mature adipocytes into progenitor cells or precursors with a double proliferative potential, which have the ability to acquire an endothelial cell phenotype:

FIG. 5a) the hDDAC cells (human dedifferentiated adipose cells) can proliferate and differentiate again into adipocytes, when they are cultured in an adipogenic medium (×400);

FIG. 5b) the hDDAC cells form branchy alignments and tubular-type structures (black arrows), when they are cultured in a medium comprising methylcellulose (×400);

FIG. 5c) the branchy alignments and tubular-type structures formed by dedifferentiation of the mature adipocytes in a culture medium containing methylcellulose are labeled with an anti-vWF antibody (×400);

FIGS. 5d) and 5e): these figures illustrate the proangiogenic properties of the hDDAC cells after they have been grafted into the hind limb rendered ischemic; the hDDAC cells are as effective as the SVF-CULT cells in restoring the angiographic score and the cutaneous blood flow in the hind limb rendered ischemic.

The values represent the mean±standard deviation, n=6 per group; *P<0.05. PBS: Mice treated with PBS. SVF: Mice treated with the SVF-CULT cells. hDDAC: Mice treated with dedifferentiated human adipocytes;

FIG. 5f) labeling of numerous CD31-positive cells forming a layer on the newly formed vessels (indicated with black arrows), with an antibody directed against the isoform of the human CD31 marker (×1000);

FIG. 6 illustrates the plasticity of the cells of the adipocyte line, for obtaining endothelial cells. The adipocyte progenitor cells have the ability to differentiate into adipocytes and to acquire a functional endothelial phenotype. The mature adipocytes can differentiate into progenitor cells with a double proliferative potential.

### DETAILED DESCRIPTION OF THE INVENTION

It should be understood that these examples are given only by way of illustration of the subject of the invention, of which they in no way constitute a limitation.

### Example 1

### Induction of a Neovascularization, by Means of Mouse SVF-CULT Cells, in a Mouse Muscle Rendered Ischemic

1.1 Materials and Methods

1.1.1 Animals and Tissue Samples

Seven-week-old male C57B1/6 or nu/nu mice (Harlan, France) are raised in a controlled environment (cycle of 12 hours of light and 12 hours of darkness at 21° C.) with free access to water and to the standard food ration. At the end of the experiments, the mice are sacrificed by cervical dislocation under anesthesia with CO₂. The inguinal adipose tissue and the muscle are rapidly removed and treated for the subsequent analyses.

1.1.2 Model of Mouse with a Hind Limb Rendered Ischemic

The animals are anesthetized by isoflurane inhalation. A ligature is applied to the right femoral artery. The mouse is subsequently injected with 10⁶ SVF-CULT cells, intramuscularly in the limb rendered ischemic.

1.1.3 Isolation of the Cells of the Adipose Tissue Stromal-Vascular Fraction and of the Bone Marrow Cells

Bone Marrow Cells:

The bone marrow cells are obtained by washing the tibias and femurs and then isolating the low-density mononuclear cells by centrifugation on a Ficoll density gradient (34).

Cells of the Extramedullary Adipose Tissue Stromal-Vascular Fraction

The cells of the stromal-vascular fraction are isolated from adipose tissue according to the protocol of Björntorp et al. (14) with minor modifications. Briefly, the mouse inguinal adipose tissue is subjected to digestion with 2 mg/ml of collagenase (Sigma) in PBS phosphate buffer containing 0.2% of BSA at 37° C. for 45 minutes. After elimination of the nonhydrolyzed fragments by filtration through a 100 μm nylon membrane, the mature adipocytes are separated from the pallets of SVF-EXT cells by centrifugation (600 g, 10 minutes).

The SVF-EXT cells are seeded at a density of 30 000 cells/cm² in DMEM F12 medium supplemented with 10% of newborn calf serum (NCS). After 6 hours of culture, the nonadherent cells are removed by washing, and then the (adherent) cells are cultured for a few days (1 to 3) before being used; SVF-CULT cells are thus obtained.

1.1.4 Quantification of the Neovascularization

The vessel density was evaluated by high-definition microangiography at the end of the treatment period (36). The angiographic score is expressed by the percentage of pixels per image that are occupied by vessels, in an area of quantification.

The microangiographic analysis is supplemented by evaluation of the capillary density using an anti-body directed against total fibronectin (36). The capillary density is then calculated in random fields of a defined area, using the Optilab/Pro software.

The functionality of the vascular network after the ischemia is analyzed by laser Doppler perfusion imaging, carried out in the mouse as described in J S Silvestre et al. (36).

1.2 Results

Firstly, the angiogenic potential of the adipose tissue was evaluated with mouse SVF-CULT cells, by comparison with bone marrow mononuclear cells.

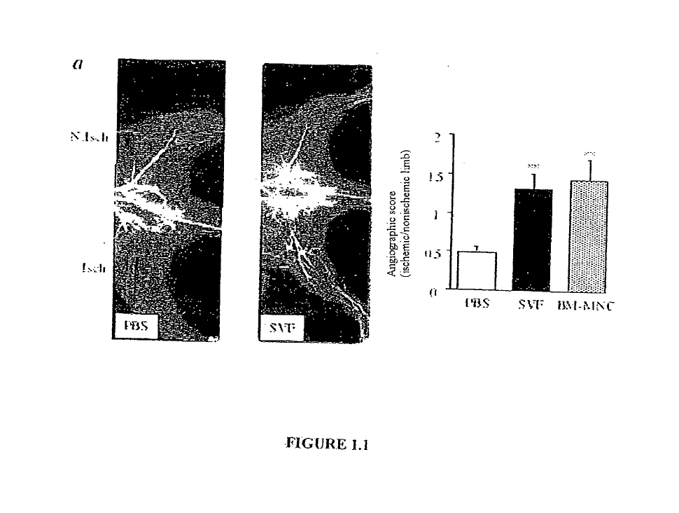

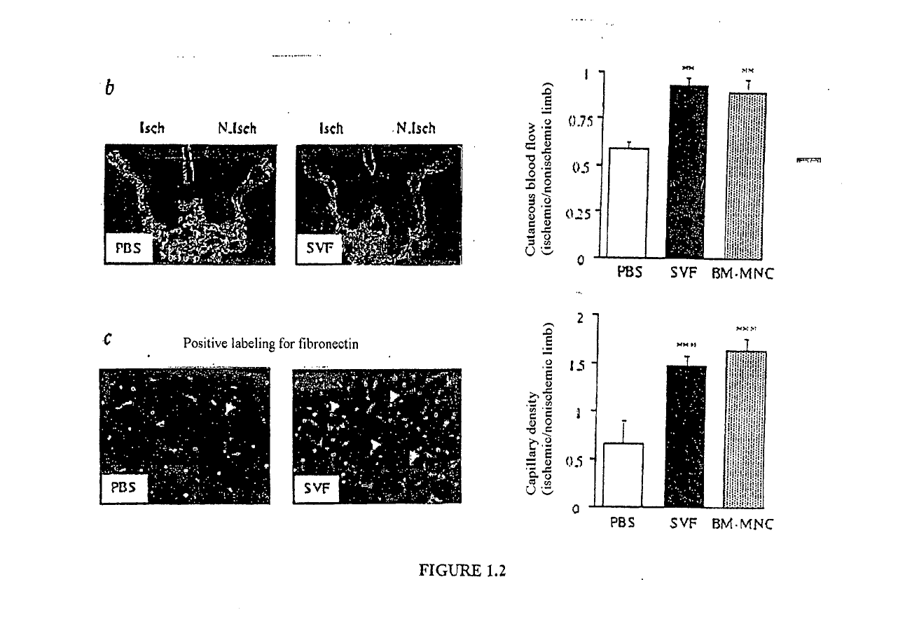

These cells are prepared from inguinal adipose tissue and placed in cultures so as to obtain a limited expansion for 1-3 days (number of successive passages limited to less than 10). The transplantation of 1×10⁶ SVF-CULT cells clearly improves the neovascularization of the tissue in hind limbs rendered ischemic, as shown by the 2.6-fold increase in the angiographic score (FIG. 1a, P<0.01), the 2.3-fold increase in the Doppler tissue perfusion score (FIG. 1b, P<0.001) and the 1.6-fold increase in the capillary density (FIG. 1c, P<0.01). The degree of neovascularization observed after the injection of 1×10⁶ SVF-CULT cells is comparable to that observed after the injection of 1×10⁶ bone marrow mononuclear cells (FIGS. 1a-c). The culture process according to the invention very significantly improves the angiogenic potential of the SVF-CULT cells, as shown by the very poor neovascularization observed after direct injection of SVF-EXT cells (not placed in culture with limited expansion as in the invention). Furthermore, experiments with cells from the vascular stroma originating from brown adipose tissue, known to be more vascularized than white tissue, proved to be fruitless.

### Example 2

### Phenotypic Characterization of the SVF-EXT Cells and of the SVF-CULT Cells

2.1 Materials and Methods

2.1.1 Preparation of SCF-EXT and SVF-CULT Cells

The mouse SVF-EXT and SVF-CULT cells are prepared as specified in Example 1.

The corresponding human cells are prepared in a similar manner, from samples of abdominal dermolipectomy or of nephrectomy containing human abdominal subcutaneous tissue, obtained with the patients' consent.

2.1.2 Phenotypic Analysis of Cells

The cells are labeled in phosphate buffered saline containing 0.2% of fetal calf serum; they are incubated with anti-mouse or anti-human monoclonal antibodies (mAbs) coupled to fluorescein isothiocyanate (FITC), to phycoerythrin (PE) or to peridinin chlorophyll protein (PerCP), for 30 minutes at 4° C. After washing, the cells are analyzed by flow cytometry (FACS Calibur, Becton Dickinson). The data obtained are then analyzed using the Cell Quest software (Becton Dickinson). All the antibodies come from BD Biosciences, with the exception of CD144, which comes from Serotec.

2.1.4 Statistical Analyses

All the statistical analyses are carried out by means of the non-paired t-test using the Prisme™ software (GraphPad software).

2.2 Results

The comparative analysis of the phenotype of the SVF-EXT and SVF-CULT (human or murine) cells was carried out by flow cytometry. Since the results obtained with the human and murine cells are comparable, only the results relating to the human cells are presented.

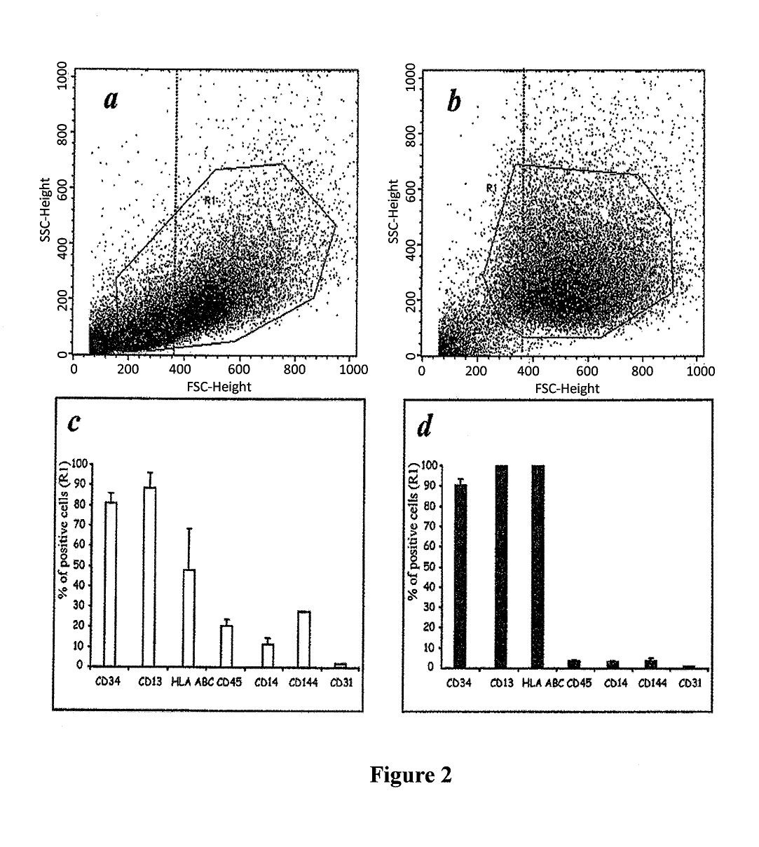

The SVF-EXT cells obtained from subcutaneous human adipose tissue are heterogeneous, as shown by the dispersion diagram in FIG. 2a. The culturing of these cells for 1-3 days under the conditions of the invention results in homogenization of the cell population, as shown by the obtaining of a single cell population, called SVF-CULT (FIG. 2b).

The antigenic phenotype confirms the dispersion diagram. The SVF-EXT cells are heterogeneous and comprise various populations, in particular hematopoietic cells (cells positive for the CD45 marker) and a population of nonhematopoietic cells (negative for the CD45 marker) expressing the markers CD34, CD13 and HLA ABC (FIG. 2c).

The stromal-vascular fraction does not contain a significant proportion of mature endothelial cells, as shown by the absence of labeling with the antibodies directed against VE-cadherin (CD144) and the CD31 marker (FIG. 2c). In the SVF-CULT population (conditions of the invention), the population is composed predominantly of undifferentiated cells, with 90+3% of cells expressing the CD34 marker and 99+0.2% of cells being positive for the CD13 and HLA ABC markers. On the other hand, these SVF-CULT cells express neither the markers characteristic of hematopoietic cells (CD45) or of monocytes/macrophages (CD14), nor the CD144 and CD31 markers, which are characteristic of differentiated endothelial cells (FIG. 2c). These results show that the cellular expansion for 1-3 days in vitro (or ex vivo) promotes the proliferation of a homogeneous population of cells (SVF-CULT cells) that possess some surface antigens characteristic of cells with proangiogenic potential, but no surface marker characteristic of differentiated cells.

### Example 3

### Induction of Neovascularization, with SVF-CULT Cells, in Mouse Muscle Rendered Ischemic, and Differentiation of this Population into Endothelial Cells

3.1 Materials and Methods

Seven-week-old male nu/nu mice (Harlan, France) are raised under the same conditions as those disclosed in Example 1.

The samples of human adipose tissue are identical to those used in Example 2.

The human and mouse SVF-CULT cells are isolated as specified in Examples 1 and 2.

The quantification of the neovascularization and the phenotypic analysis are carried out as specified, respectively, in Examples 1 and 2.

3.2 Results

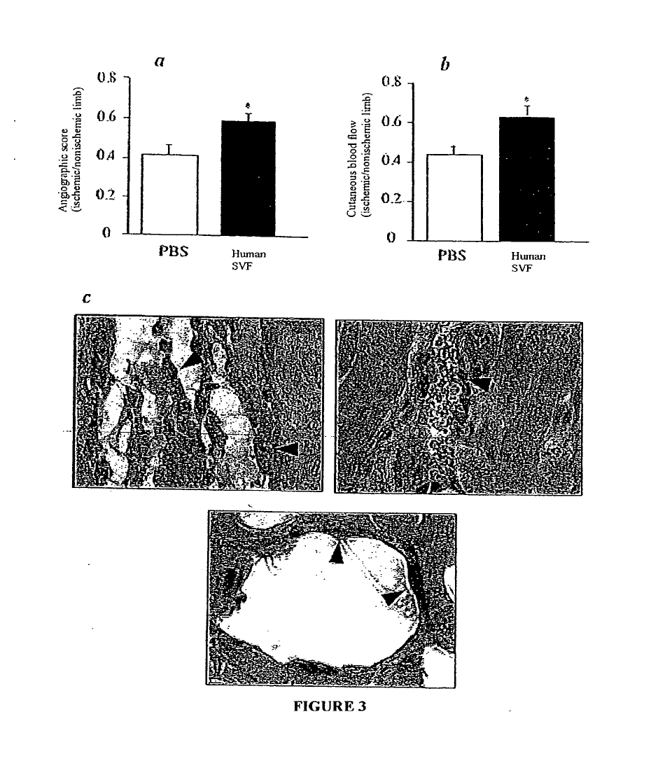

The effect of the injection (or transplantation) of the human SVF-CULT cells on revascularization is evaluated in immunodeficient Nude mice. As for the mouse SVF-CULT cells, the injection of 1×10⁶ human SVF-CULT cells after 15 days of ischemia of the hind limbs makes it possible to obtain a significant increase in the angiographic score and in the cutaneous blood flow (by a factor, respectively, of 1.6 and 1.5 when compared with the Nude mice rendered ischemic and not treated, P<0.01) (FIGS. 3a and 3b). Two possible mechanisms, which are not incompatible, may explain the proangiogenic effects: the release of angiogenic growth factors by the SVF-CULT cells or a direct contribution of the injected cells by incorporation (or transplantation) of the latter into the regenerated vessels. In fact, VEGF is detected as being a potential angiogenic factor (31+8 ng/ml).

Thus, in order to evaluate the ability of the SVF-CULT cells to be incorporated into new blood vessels, immunochemistry experiments were carried out using an antibody specific for the human CD31 marker, which does not react with mouse tissue. Numerous cells positive for the CD31 marker forming a layer on the regenerated vessels are demonstrated in the treated hind limb (FIG. 3c). No cell positive for the CD31 marker is detected in the other hind limb which is not treated. The detection of human CD31+ cells strongly suggests that, under in vivo conditions, the SVF-CULT cells differentiate into endothelial cells and contribute directly to the vessel regeneration.

### Example 4

### Spontaneous Differentiation of Human SVF-CULT Cells into Adipocytes or into Endothelial Cells, In Vitro or In Vivo in the Matrigel® Matrix

4.1 Materials and Methods

The human cells of the extramedullary stromal-vascular fraction (SVF) are prepared and placed in culture as in Example 2.

To test their potential for differentiation in vitro at the clonal level while preserving cellular function, the SVF-CULT cells are placed in culture in semi-solid medium (methylcellulose; 15). A primary culture of SVF-CULT cells is trypsinized, and then seeded at a concentration of 7×10³ cells/ml into 1.5 ml of Methocult MG3534, MG, H4534 (StemCell Technologies) or any other equivalent medium. The cells are cultured for 10 days in order to stimulate their development in terms of cells having an endothelial-type morphology, and then analyzed by immunolabeling. The colonies of the cultures in the presence of methylcellulose are washed with PBS buffer and fixed in a methanol/acetone mixture for 20 minutes at −20° C. The preparations are then blocked in PBS containing 1% BSA, and incubated for 1 hour with either anti-human CD31 antibodies (Dako, reference M0823) or anti-human vWF factor or anti-mouse vWF factor antibodies.

The angiogenesis assay, in vivo, using the Matrigel® matrix, is carried out in the following way: the mice are given a subcutaneous injection of a volume of 0.5 ml of Matrigel® matrix containing 10⁶ SVF-CULT cells isolated from mouse tissue or from human tissue. On the 14th day, the mice are sacrificed and the angiogenesis is analyzed as described in R. Tamarat et al. (37). For the immunolabeling, the Matrigel® matrices are treated as described in N. Nibbelink et al. (35). Sections 5 μm thick are stained with alkaline phosphatase (BCIP/NBT) after having been incubated with an alkaline phosphatase-coupled antibody from Jackson, or else they are stained with diaminobenzidine (DAB) after having been incubated with a primary antibody and then with a biotinylated secondary antibody (Dako Carpinteria, CA); the anti-human 0× Phos complex IV antibody comes from Molecular Probes (Eugene, Oreg., USA).

By way of comparison, SVF-CULT cells are cultured in an adipogenic medium (Björntorp et al., mentioned above).

4.2 Results

The differentiation of the SVF-CULT cells was analyzed in vitro, in a semi-solid medium that makes it possible to study cell differentiation at the clonal level while preserving cell function (methylcellulose), and in vivo after injection of cells associated with a solid matrix (Matrigel®)

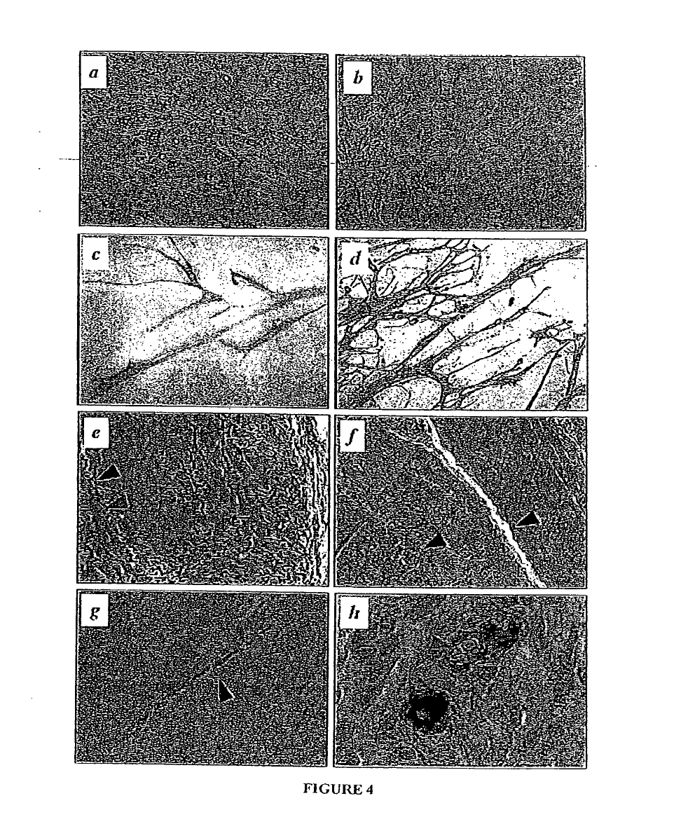

Under these conditions, the SVF-CULT cells form a network having a structure in the form of hollow tubes (FIG. 4b). Antibodies directed, respectively, against the CD31 marker and against the von Willebrand (vWF) factor strongly label the SVF-CULT cells (FIGS. 4c and 4d). When the SVF-CULT cells are injected in combination with a Matrigel® matrix, the cells form numerous tubular-type structures within the Matrigel® matrix. The presence of erythrocytes in the lumen of these tubular-type structures demonstrates the existence of a functional vascular structure (FIGS. 4e and f). The antibodies directed against the CD31 marker and against the vWF marker positively label these structures resembling vessels (FIGS. 4g and h).

By comparison, the SVF-CULT cells cultured in an adipogenic medium differentiate into adipocytes (FIG. 4a).

All these results show that the SVF-CULT cells spontaneously exhibit the phenotypic and functional properties of endothelial progenitor cells.

### Example 5

### Dedifferentiation of Mature Human Adipocytes in Culture

5.1 Materials and Methods

Dedifferentiation of Mature Human Adipocytes

The mature human adipocyte fraction, isolated from a sample of adipose tissue as described in Example 1, is washed carefully in DMEM-F12 medium supplemented with 10% of NCS and prepared in the form of a suspension at a concentration of 10⁶ cells/ml. A sample of 100 μl of the cell suspension is transferred onto a 25 mm Thermanox coverslip and placed in a 35 mm culture dish. The first coverslip is covered with a second, and, after incubation for 15 minutes at ambient temperature, 1.5 ml of DMEM F12 supplemented with 10% of NCS are added. After 4 or 5 days of incubation, the adherent cells containing small lipid droplets (cells of preadipocyte type) appear; they become modified into a fibroblast-type morphology devoid of lipid droplets (hDDAC cells for human dedifferentiated adipose cells).

These fibroblastic-type cells then begin to actively divide and can undergo several passages without major modification of their characteristics.

The dedifferentiated human adipocytes are placed in culture in methylcellulose and analyzed by immunolabeling as described in Example 4. Furthermore, their angiogenic potential is analyzed in vivo, after injection in a Matrigel® matrix, as described in Example 4. The angiogenic potential of the SVF-CULT cells prepared as described in Example 3 is analyzed in parallel.

Alternatively, the dedifferentiated human adipocytes are placed in culture in adipogenic medium (Björntorp et al., mentioned above).

5.2 Results

In order to obtain a homogeneous population of adipocyte precursor cells from adipose tissue and to confirm the existence of a precursor common to adipocytes and to endothelial cells derived from the SVF-CULT cells, mature adipocytes were dedifferentiated, according to previously described protocols (16; 17; 18; 19). The mature adipocytes isolated from adipose tissue represent 99% of a population of floating cells. The only cellular contamination comes from macrophages rich in lipid droplets, with a ratio of a few contaminating cells per 1000 cells. When the adipocytes are placed in culture under the above-mentioned conditions (17), they initially lose their fatty acids and change their morphology to preadipocyte-type cells and then to fibroblast-type cells which can attach to the coverslip. This morphological change is associated with functional changes, given that the adipocytes also lose their enzymatic content for lipolysis and lipogenesis and also the molecular markers (17).

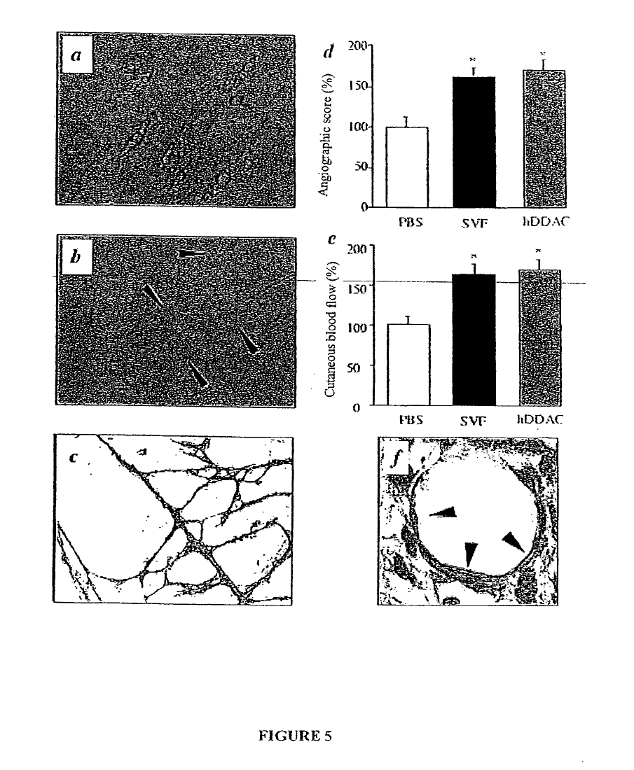

The homogeneous population of human dedifferentiated adipocytes (hDDACs) have the ability to proliferate and to differentiate again into adipocytes, when it is cultured in an adipogenic medium (FIG. 5a).

The same homogeneous population of human dedifferentiated adipocytes (hDDACs) cultured in a medium containing methylcellulose forms branchy alignments and structures in the form of a tube (FIG. 5b) and coexpresses, at more than 99%, the same markers as the SVF-CULT cells (CD13, CD34 and HLA ABC), including the vWF marker (FIG. 5c).

As is the case for the SVF-CULT cells, when the hDDAC cells are injected in association with the Matrigel® matrix, they form numerous tubular-type structures, which contain erythrocytes in their lumen, demonstrating the existence of a functional vascular structure.

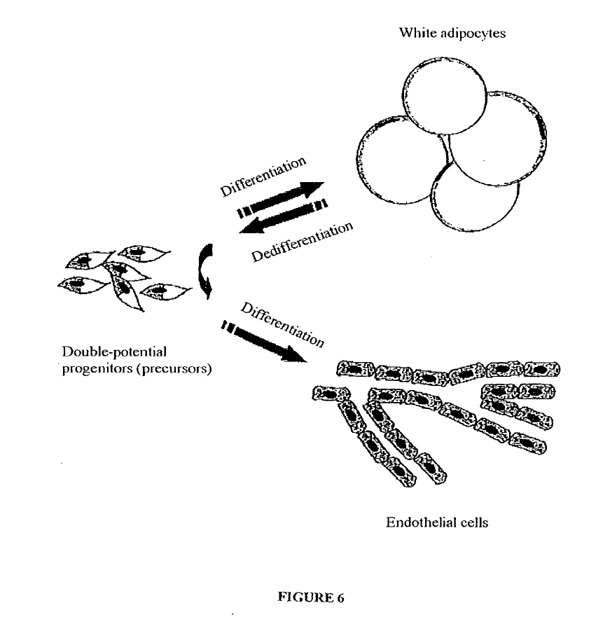

These results are illustrated in FIG. 6, which illustrates the plasticity of the cells of the adipocyte line, for obtaining endothelial cells. The adipocyte progenitor cells have the ability to differentiate into adipocytes and to acquire a functional endothelial phenotype. The mature adipocytes can dedifferentiate into progenitor cells with a double proliferative potential.

### Example 6

### Stimulation of Neovascularization, with Human Dedifferentiated Adipocytes, in Mouse Muscle Rendered Ischemic, and Differentiation of these Adipocytes into Endothelial Cells

6.1 Materials and Methods

The angiogenic potential of the hDDAC cells was analyzed in Nude mice as for the SV-CULT cells (Example 3), which serve as comparison.

6.2 Results

The hDDAC cells are as effective as the SVF-CULT cells in restoring the vascularization of the hind limbs rendered ischemic (FIGS. 5d and 5e). As is the case for the SVF-CULT cells, numerous cells positive for the CD31 marker are identified, which form a layer on the newly formed vessels of the hind limb, into which the hDDAC cells were injected (FIG. 5f).

### Example 7

### Use of the SVF-CULT Cells to Induce Neovascularization in an Atheromatous Context (Murine ApoE −/− Model)

7.1 Materials and Methods

The angiogenic potential of the SVF-CULT cells was analyzed in 14-week-old ApoE deficient mice (ApoE Knock-out (ApoE KO or ApoE −/−); Iffa-Credo), as in the C57B1/6 mouse (Example 1). The angiogenic potential of bone marrow mononuclear cells, in ApoE KO mice, is analyzed in parallel, by way of comparison. The control group is given an injection of PBS, under the same conditions.

More specifically, the neovascularization process was analyzed by laser Doppler microangiography, 4 weeks after femoral occlusion. The statistical analysis was carried out by means of an ANOVA-type variance test for comparing each parameter (n=6 for each group). A Bonferroni t test subsequently made it possible to identify the groups causing these differences. A value of P<0.05 is considered to be significant.

7.2 Results

The administration of SVF-CULT cells increases the angiographic score by a factor of 2 (p<0.01) and the blood flow by a factor of 1.5 (p<0.01), in the hind limb rendered ischemic of the treated ApoE KO mice, compared with the nontreated ApoE KO mice (Table I). The angiogenic potential of the SVF-CULT adipose cells is similar to that of the bone marrow mononuclear cells (Table I).

TABLE I
Angiogenic potential of the SVF-CULT cells in ApoE −/− mice

| | Angiographic score* | Cutaneous blood flow* |
|---|---|---|
| | (limb rendered | (limb rendered |
| | ischemic/limb not | ischemic/limb not |
| Treatment | rendered ischemic) | rendered ischemic) |
| SVF-CULT | 0.982 ± 0.3 | 0.963 ± 0.04 |
| BM-MNC | 1.002 ± 0.04 | 0.995 ± 0.03 |
| PBS | 0.47 ± 0.02 | 0.65 ± 0.03 |

*The values represent the mean ± standard deviation on a group of 6 animals

The treatment of the limb rendered ischemic of the ApoE (−/−) mice is effective and promotes angiogenesis/neovascularization. This effect is as effective as the injection of bone marrow mononuclear cells. The SVF-CULT cells can serve their proangiogenic potential in an atheromatous context.

### Example 8

### Improvement in the Angiogenic Potential of the SVF-CULT Cells by Modification of their Redox State

8.1 Materials and Methods

The angiogenic potential of the SVF-CULT cells treated, in vitro, with antimycin (40 nM) and/or pyrrolidine dithiocarbamate (PDTC; 0.5 mM) two days before the injection, was analyzed in the model of the mouse with a hind limb rendered ischemic, as described in Example 1. Furthermore, after the injection of the SVF-CULT cells treated with antimycin alone, by adding antimycin to the culture medium, or not treated, the mice were or were not given a daily i.p. injection of antimycin (50 μl at 40 nM). The mice treated similarly with PDTC alone or in combination with antimycin receive no treatment after the injection of cells.

The angiogenic potential of the nontreated SVF-CULT cells, in mice not treated after the injection of the cells, was analyzed in parallel, by way of comparison. The control group was given an injection of ethanol, under the same conditions.

The neovascularization process was analyzed by microangiography and, optionally, by laser Doppler, 8 days after femoral occlusion. The statistical analysis was carried out by means of an ANOVA-type variance test for comparing each parameter (n=5 for each group). A Bonferroni t test subsequently made it possible to identify the groups causing these differences. A value of P<0.05 is considered to be significant.

8.2 Results

The effect of the modification of the redox state of the SVF-CULT cells on their proangiogenic potential was tested with a mitochondrial respiratory chain complex III inhibitor which induces the production of active oxygen species and a modification of the redox state of cells (antimycin), and an antioxidant which limits the production of active oxygen species and the cellular redox state (PDTC: pyrrolidine dithiocarbamate). The results are given in Tables II and III below.

TABLE II
Effect of the treatment in vitro or in vivo, of the SVF-CULT cells with
antimycin, on the angiogenic potential of the cells

| | Blood flow in the limb |
|---|---|
| | rendered ischemic/limb |
| Injection | not rendered ischemic |
| Ethanol | 0.430 ± 0.025 |
| SVF-CULT cells | 0.616 ± 0.062 |
| SVF-CULT cells treated with | 0.830 ± 0.031 |
| antimycin then antimycin (i.p.) | |
| SVF-CULT cells not treated, then | 0.426 ± 0.022 |
| antimycin (i.p.) | |

TABLE III
Effect of the treatment, in vitro, of the SVF-CULT cells with antimycin
and/or PDTC, on the angiogenic potential of the cells

| | | Angiographic |
|---|---|---|
| | Blood flow in the | score in the |
| | limb rendered | limb rendered |
| | ischemic/limb not | ischemic/limb not |
| Injection | rendered ischemic | rendered ischemic |
| Ethanol | 0.430 ± 0.025 | 0.588 ± 0.033 |
| SVF-CULT cells | 0.690 ± 0.014 | 0.844 ± 0.027 |
| SVF-CULT cells | 0.882 ± 0.015 | 1.094 ± 0.030 |
| treated with | | |
| antimycin | | |
| SVF-CULT cells | 0.716 ± 0.024 | 0.846 ± 0.026 |
| treated with | | |
| antimycin and PDTC | | |
| SVF-CULT cells | 0.718 ± 0.041 | 0.774 ± 0.023 |
| treated with PDTC | | |

The treatment of the SVF-CULT cells with antimycin alone, before injection into the limb rendered ischemic, has a significantly positive and substantial effect on revascularization, as shown by the 1.4-fold increase in blood flow (p<0.05; Tables II and III) and the 1.3-fold increase in the angiographic score (p=0.06; Table III). This effect is prevented by an antioxidant (Table III), which indicates the involvement of active oxygen species and/or a modification of the redox state in the effects favorable to angiogenesis. On the other hand, antimycin has no effect when it is administered directly to the animal, after the injection of the SVF-CULT cells into the muscle rendered ischemic (Table II).

### BIBLIOGRAPHICAL REFERENCES

1. Ailhaud G. et al., Annu. Rev. Nutr., 1992, 12, 207-33.
2. Castellot J. J. et al., Proc. Natl. Acad. Sci. USA, 1982, 79, 5597-601.
3. Bouloumie A. et al., Ann. Endocrinol. (Paris), 2002, 63, 91-5.
4. Wasserman P., The development of adipose tissue, 1965.
5. Dobson D. E. et al., Cell, 1990, 61, 223-30.
6. Claffey K P. et al., J. Biol. Chem., 1992, 267, 16316-22.
7. Bouloumie A. et al., Circ. Res., 1998, 83, 1059-66.
8. Sierra-Honigmann M. R. et al., Science, 1998, 281, 1683-6.
9. Rupnick M. A. et al., Proc. Natl. Acad. Sci. USA, 2002, 99, 10730-5.
10. Zuk P A. et al., Mol. Biol. Cell, 2002, 13, 4279-95.
11. Erickson G R. et al., Biochem. Biophys. Res. Commun., 2002, 290, 763-9.
12. Cousin B. et al., Biochem. Biophys. Res. Commun., 2003, 301, 1016-22.
13. Safford K M. et al., Biochem. Biophys. Res. Commun., 2002, 294, 371-9.
14. Björntorp P. et al., J. Lipid Res., 1978, 19, 316-24.
15. Gehling U. M. et al., Blood, 2000, 95, 3106-12.
16. Negrel R. et al., Proc. Natl. Acad. Sci. USA, 1978, 75, 6054-8.
17. Negrel R. et al., Methods Enzymol., 1985, 109, 377-385.
18. Aoki S. et al., Cell Struct. Fund., 2003, 28, 55-60.
19. Shigematsu M. et al., Cell. Struct. Funct., 1999, 24, 89-100.
20. Fernandez Pujol B. et al., Differentiation, 2000, 65, 287-300.
21. Rehman J. et al., Circulation, 2003, 107, 1164-9.
22. Jiang Y. et al., Nature, 2002, 418, 41-9.
23. Jiang Y. et al., Exp. Hematol., 2002, 30, 896-904.
24. Gronthos S. et al., J. Cell Physiol., 2001, 189, 54-63.
25. Carmeliet P., Nat. Med., 2003, 9, 653-60.
26. Orlic D. et al., Nature, 2001, 410, 701-705.
27. Kocher A A. et al., Nat. Med., 2001, 7, 430-436.
28. Assmus B. et al., Circulation, 2002, 106, 3009-17.
29. Strauer B. E. et al., Circulation, 2002, 106, 1913-8.
30. Tateishi-Yuyama E. et al., Lancet, 2002, 360, 427-35.
31. Tse H. F. et al., Lancet, 2003, 361, 47-9.
32. Stamm C. et al., Lancet, 2003, 361, 45-6.
33. Charrière G. et al., J. Biol. Chem., 2003, 278, 9850-5.
34. Mallat Z. et al., Circ. Res., 2002, 91, 441-8.
35. Nibbelink M. et al., J. Biol. Chem., 2001, 276, 47291-5.
36. Silvestre J. S. et al., Circ. Res., 2001, 89, 259-64.
37. Tamarat R. et al., Lab. Invest., 2002, 82, 747-56.
38. Luttun A. et al., Trends Cardiovasc. Med., 2002, 12, 88-96.
39. Iba O. et al., Circulation, 2002, 106, 2019-2025.
40. Benelli R et al., Internat. J. Biol. Markers, 1999, 14, 4, 243-246.

1. A method for totally or partially reconstructing a functional vascular network in a subject comprising administering to said subject a homogenous population of cells derived from medullary or extramedullary white adipose tissue, wherein said cells express at least the surface antigens CD13 and HLA ABC.

2. The method as claimed in claim 1, wherein said cells also express the surface antigen CD34.

3. The method as claimed in claim 1, wherein said cells are obtained by limited cellular expansion in culture.

4. The method as claimed in claim 3, wherein said cells are obtained by a limited cellular expansion with less than 10 successive passages of said cells.

5. The method as claimed in claim 1, wherein said cells are derived from mature dedifferentiated adipocytes.

6. The method as claimed in claim 1, wherein said cells are associated with a solid or semi-solid polymeric support.

7. The method as claimed in claim 6, wherein said solid polymeric support is selected from the group consisting of reconstituted basal membrane matrices comprising at least one of the following elements: collagen, laminin and proteoglycans, and reconstituted extracellular matrices comprising one of the following elements: fibronectin, collagen, laminin and thrombospondin.

8. The method as claimed in claim 6, wherein said polymeric support comprises enzymes that degrade said matrices, enzymatic inhibitors, and growth factors.

9. The method as claimed in claim 6, characterized in that said semi-solid polymeric support is a cellulose derivative.

10. The method as claimed in claim 1, wherein said cells are genetically modified.

11. The method as claimed in claim 10, wherein said cells comprise at least one mutation of an autologous gene.

12. The method as claimed in claim 10, wherein said cells contain at least one copy of a heterologous gene.

13. The method as claimed in claim 10, wherein said cells are of human origin.

14. The method as claimed in claim 1, wherein said cells are associated with at least one vehicle and/or one support that is suitable for parenteral or intra-site administration.

15-19. (canceled)

20. The method as claimed in claim 9, wherein said support is methylcellulose.

21. The method as claimed in claim 1, wherein said cells are administered parenterally or intrasite.

22. A method for preparing a medicinal product intended for the total or partial reconstruction of a functional vascular network, comprising isolating a homogenous population of cells from medullary or extramedullary white adipose tissue, wherein said cells express at least the surface antigens CD13 and HLA ABC, and incorporating the isolated cells into a medicinal product together with at least one vehicle and/or one support which is suitable for parenteral or intra-site administration.

23. The method as claimed in claim 18, wherein said cells also express the surface antigen CD34.

24. The method as claimed in claim 22, wherein said cells obtained by limited cellular expansion in culture.

25. The method as claimed in claim 24, wherein said cells are obtained by a limited cellular expansion of less than 10 successive passages.

26. The method as claimed in claim 22, wherein said cells are derived from mature dedifferentiated adipocytes.

27. The method as claimed in claim 22, wherein said cells are incorporated into a medicinal product with a solid or semi-solid polymeric support.

28. The method as claimed in claim 27, wherein said polymeric support is selected from the group consisting of reconstituted basal membrane matrices comprising at least one of the following elements: collagen, laminin and proteoglycans, and reconstituted extracellular matrices comprising one of the following elements: fibronectin, collagen, laminin and thrombospondin.

29. The method as claimed in claim 28, wherein said polymeric support comprises enzymes that degrade said matrices, enzymatic inhibitors, and growth factors.

30. The method as claimed in claim 27, wherein said semi-solid polymeric support is a cellulose derivative.

31. The method as claimed in claim 22, wherein said cells are genetically modified.

32. The method as claimed in claim 31, wherein said cells comprise at least one mutation of an autologous gene.

33. The method as claimed in claim 31, wherein said cells contain at least one copy of a heterologous gene.

34. The method as claimed in claim 31, wherein said cells are of human origin.
